(19) United States
(12) Patent Application Publication
(10) Pub. No.: US 2018/0353066 A1
(43) Pub. Date: Dec. 13, 2018

(54) IMAGE PROCESSING APPARATUS AND IMAGE PROCESSING METHOD

(71) Applicant: CANON KABUSHIKI KAISHA, Tokyo (JP)

(72) Inventors: Akihito Uji, Kyoto-shi (JP); Hiroshi Imamura, Kawasaki-shi (JP); Tomoyuki Makihira, Tokyo (JP); Nagahisa Yoshimura, Kyoto-shi (JP)

(21) Appl. No.: 15/780,915

(22) PCT Filed: Nov. 25, 2016
(86) PCT No.: PCT/JP2016/004961

§ 371 Date: Jun. 1, 2018

(30) Foreign Application Priority Data
Dec. 3, 2015 (JP) 2015-236742

(51) Int. Cl. A61B 3/117; A61B 3/10; A61B 3/00; G06T 11/00; G06T 3/00; G06T 7/00; G06T 7/50; A61B 3/16

(52) U.S. Cl. CPC A61B 3/117; A61B 3/102; A61B 3/0041; G06T 11/003; G06T 3/0037; G06T 2207/30041; G06T 7/50; A61B 3/0025; A61B 3/16; G06T 2207/10101; G06T 2207/10028; G06T 7/0012

(57) ABSTRACT

An image processing apparatus includes an acquisition unit configured to acquire a tomographic image containing an aqueous humor outflow pathway region that includes at least one of a Schlemm's canal and a collector channel in an anterior ocular segment of a subject's eye, and a generation unit configured to generate an image with the aqueous humor outflow pathway region emphasized or extracted therein based on a luminance value in the tomographic image.

### TECHNICAL FIELD

The present invention relates to an image processing apparatus and an image processing method that process a tomographic image of a subject's eye.

### BACKGROUND ART

Tomographic image capturing apparatuses for an ocular portion, such as an optical coherence tomography (OCT), enable three-dimensional observation of a state inside a retinal layer. Such tomographic image capturing apparatuses have been widely used in ophthalmologic care since they are useful to diagnose a disease more accurately. One example of types of the OCT is a time domain OCT (TD-OCT), which is composed of a combination of a wideband light source and a Michelson interferometer. The TD-OCT is configured to measure interference light with backscattered light of a signal arm to acquire information indicating a depth resolution, by scanning a delay of a reference arm. However, the TD-OCT configured in this manner requires mechanical scanning, and thus it is difficult to acquire an image at a high speed with use of the TD-OCT. As such, a spectral domain OCT (SD-OCT) configured to use a wideband light source and acquire an interference signal with use of a spectrometer has been used as a method for acquiring an image at a higher speed. In recent years, a swept source OCT (SS-OCT) configured to temporally disperse light by using a high-speed wavelength sweeping light source having a wavelength of 1 μm as a central wavelength of the light source has been developed, which has enabled acquisition of a tomographic image with a further wide angle of view and further deep penetration. Although an anterior ocular segment includes an opaque tissue such as a sclera, a three-dimensional tomographic image of the anterior ocular segment that contains the sclera can be acquired with use of a light source having a central wavelength of 1 μm. The tomographic image of the anterior ocular segment captured by the SS-OCT can be used for, for example, diagnosis and treatment planning/follow-up monitoring of glaucoma and a corneal disease. In this regard, R. Poddar et al. (“Three-dimensional anterior segment imaging in patients with type I Boston Keratoprosthesis with switchable full depth range swept source optical coherence tomography”, Journal of Biomedical Optics 18 (8), August 2013) discusses a technique of acquiring a tomographic image containing a Schlemm's canal by capturing an image of a junction between a cornea and the sclera with use of an SS-OCT equipped with a light source having a central wavelength of 1 μm and operable at an A-scan rate of 100 kHz.

### CITATION LIST

### Non Patent Literature

NPL 1: R. Poddar et al., “Three-dimensional anterior segment imaging in patients with type 1 Boston Keratoprosthesis with switchable full depth range swept source optical coherence tomography”, Journal of Biomedical Optics 18 (8), August 2013

NPL 2: R. Poddar et al., “In vivo volumetric depth-resolved vasculature imaging of human limbus and sclera with 1 μm swept source phase-variance optical coherence angiography”, Journal of Optics (J. Opt.) 17 (6), June 2015

### SUMMARY OF INVENTION

### Solution to Problem

According to an aspect of the present invention, an image processing apparatus includes an acquisition unit configured to acquire a tomographic image containing an aqueous humor outflow pathway region that includes at least one of a Schlemm's canal and a collector channel in an anterior ocular segment of a subject's eye, and a generation unit configured to generate an image with the aqueous humor outflow pathway region emphasized or extracted therein based on a luminance value of the tomographic image.

According to another aspect of the present invention, an image processing method includes acquiring a tomographic image containing an aqueous humor outflow pathway region that includes at least one of a Schlemm's canal and a collector channel in an anterior ocular segment of a subjects eye, and generating an image with the aqueous humor outflow pathway region emphasized or extracted therein based on a luminance value of the tomographic image.

Further features of the present invention will become apparent from the following description of exemplary embodiments with reference to the attached drawings.

### BRIEF DESCRIPTION OF DRAWINGS

FIG. 1 is a block diagram illustrating a configuration of an image processing system according to first and second exemplary embodiments of the present invention.

FIG. 2A illustrates an anatomical structure of an anterior ocular segment.

FIG. 2B illustrates the anatomical structure of the anterior ocular segment.

FIG. 3A is a flowchart illustrating processing performed by the image processing system according to the exemplary embodiments of the present invention.

FIG. 3B is a flowchart illustrating processing performed by an image processing system according to an exemplary embodiment of the present invention.

FIG. 4A illustrates a content of image processing according to the first and second exemplary embodiments of the present invention.

FIG. 4B illustrates the content of the image processing according to the first and second exemplary embodiments of the present invention.

FIG. 4C illustrates the content of the image processing according to the first and second exemplary embodiments of the present invention.

FIG. 4D illustrates the content of the image processing according to the first and second exemplary embodiments of the present invention.

FIG. 4E illustrates the content of the image processing according to the first and second exemplary embodiments of the present invention.

FIG. 4F illustrates the content of the image processing according to the first and second exemplary embodiments of the present invention.

FIG. 4G illustrates the content of the image processing according to the first and second exemplary embodiments of the present invention.

FIG. 4H illustrates the content of the image processing according to the first and second exemplary embodiments of the present invention.

FIG. 4I illustrates the content of the image processing according to the first and second exemplary embodiments of the present invention.

FIG. 4J illustrates the content of the image processing according to the first and second exemplary embodiments of the present invention.

FIG. 4K illustrates the content of the image processing according to the first and second exemplary embodiments of the present invention.

FIG. 5A illustrates the content of the image processing according to the first and second exemplary embodiments of the present invention.

FIG. 5B illustrates the content of the image processing according to the first and second exemplary embodiments of the present invention.

FIG. 5C illustrates the content of the image processing according to the first and second exemplary embodiments of the present invention.

FIG. 5C illustrates the content of the image processing according to the first and second exemplary embodiments of the present invention.

FIG. 5E illustrates the content of the image processing according to the first and second exemplary embodiments of the present invention.

FIG. 6A is a flowchart illustrating details of processing performed in step S330 in the first or second exemplary embodiment of the present invention.

FIG. 6B is a flowchart illustrating details of processing performed in step S330 in the first or second exemplary embodiment of the present invention.

FIG. 7 is a block diagram illustrating a configuration of an image processing system according to a third exemplary embodiment of the present invention.

FIG. 8A is a flowchart illustrating details of processing performed in step S341 or S351 in the third exemplary embodiment of the present invention.

FIG. 8B is a flowchart illustrating details of processing performed in step S341 or S351 in the third exemplary embodiment of the present invention.

FIG. 8C is a flowchart illustrating details of processing performed in step S341 or S351 in the third exemplary embodiment of the present invention.

FIG. 9A illustrates a content of image processing according to the third exemplary embodiment of the present invention.

FIG. 9B illustrates the content of the image processing according to the third exemplary embodiment of the present invention.

FIG. 9C illustrates the content of the image processing according to the third exemplary embodiment of the present invention.

### DESCRIPTION OF EMBODIMENTS

In general, a less invasive treatment (an aqueous humor outflow pathway reconstruction surgery) is administered for a glaucomatous eye. In the less invasive treatment, an intraocular pressure is reduced by recovering a flow amount of aqueous humor passing through the Schlemm's canal through, for example, an incision of a trabecular meshwork adjacent to a Schlemm's canal. For the aqueous humor outflow pathway reconstruction surgery, a measure for non-invasively evaluating patency (no occurrence of stenosis and occlusion) of an aqueous humor outflow pathway connected to a surgical site (the trabecular meshwork), i.e., a Schlemm's canal region SC, a collector channel region CC, a deep scleral venous plexus DSP, an intrascleral venous plexus ISP, and episcleral veins EP, is required.

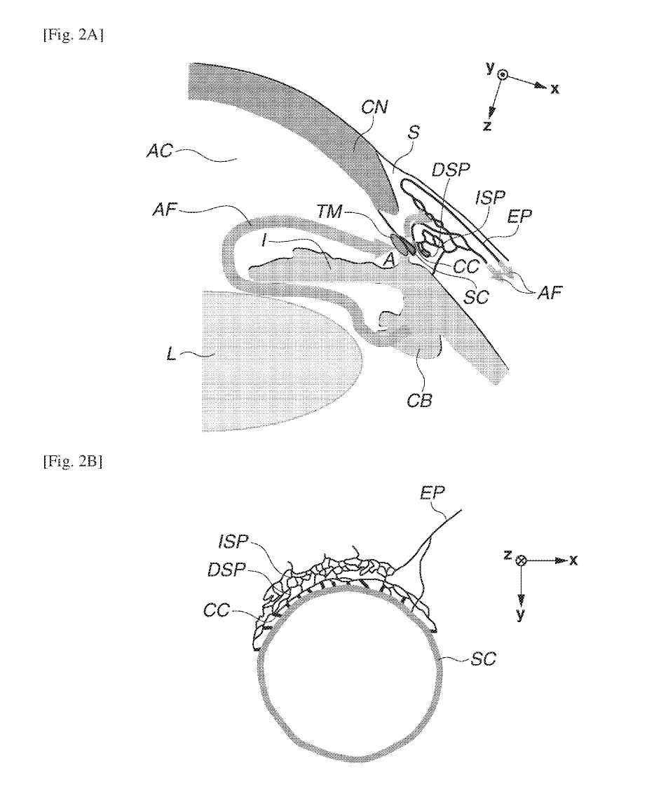

Now, anatomy of an anterior ocular segment and a pathway of outflow of aqueous humor AF will be described with reference to FIGS. 2A and 2B. As illustrated in FIG. 2A, the anterior ocular segment includes a cornea CN, a sclera S, a lens L, an iris I, a ciliary body CB, an anterior chamber AC, an angle A, and the like. The aqueous humor AF produced in the ciliary body CB passes through between the iris I and the lens L, travels through the anterior chamber AC, and enters the Schlemm's canal region SC via a trabecular meshwork TM. Then, the aqueous humor AF flows in veins in the sclera S via the collector channel region CC and is drained. The veins in the sclera S run from a deep layer (a deep side connected to the collector channel region CC) of the sclera S to a front layer side of the sclera S in an order of the deep scleral venous plexus DSP, the intrascleral venous plexus ISP, and the episcleral veins EP.

Further, as viewed from a front side as illustrated in FIG. 2B, the Schlemm's canal region SC (gray) runs in such a way as to encircle an outer side of a periphery of the cornea CN, and a plurality of collector channel regions CC (black) branches off from the Schlemm's canal region SC and is further connected to the deep scleral venous plexus DSP.

In the aqueous humor outflow pathway reconstruction surgery, it is necessary to determine a surgical site that can be expected to ensure the recovery of the flow amount of the aqueous humor and the reduction in the intraocular pressure. Thus, it is desired to non-invasively figure out which collector channel region CC and which vein in the sclera connected thereto maintain the patency, and then select the trabecular meshwork TM (or the Schlemm's canal region SC) as close to the patent collector channel region CC as possible, as a treatment site. Accordingly, it becomes necessary to attain the measure for non-invasively figuring out the patency of the aqueous humor outflow pathway through the Schlemm's canal region SC and subsequent thereto.

As such, the present invention is directed to enabling a user to know whether there is stenosis, occlusion, or the like in an aqueous humor outflow pathway region including at least one of the Schlemm's canal region SC and the collector channel region CC in a tomographic image of the anterior ocular segment.

Therefore, image processing apparatuses according to the present exemplary embodiments each include an acquisition unit configured to acquire a tomographic image containing the aqueous humor outflow pathway region including at least one of the Schlemm's canal region SC and the collector channel region CC in the anterior ocular segment of a subject's eye.

Further, the image processing apparatuses according to the present exemplary embodiments each include a generation unit configured to generate an image with the aqueous humor outflow pathway region emphasized or extracted therein based on a luminance value in the tomographic image.

With this configuration, according to the present exemplary embodiments, the image processing apparatuses can non-invasively emphasize or extract the aqueous humor outflow pathway region including at least one of the Schlemm's canal region SC and the collector channel region CC with use of the tomographic image of the anterior ocular segment, which enables the user to know whether there is stenosis, occlusion, or the like in the aqueous humor outflow pathway region.

### First Exemplary Embodiment

An image processing apparatus according to a first exemplary embodiment of the present invention will be described below. The image processing apparatus performs processing for differentiating a luminance value in a depth direction on the tomographic image of the anterior ocular segment that contains at least a deep scleral portion. Next, the image processing apparatus performs projection processing based on an amount of a variation in the luminance value in the depth direction with respect to different depth ranges of this differential image, thereby generating a group of projection images in which the aqueous humor outflow pathway region including the Schlemm's canal region SC and the collector channel region CC is emphasized in the different depth ranges. Further, the image processing apparatus binarizes each of these projection images based on a predetermined threshold value, thereby extracting a two-dimensional aqueous humor outflow pathway region.

(Overall Configuration of Image Processing Apparatus)

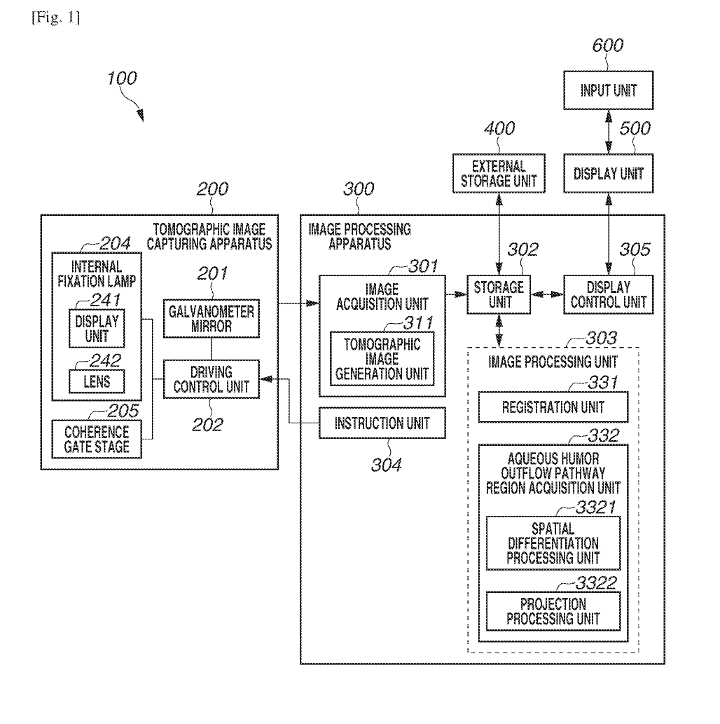

In the following description, an image processing system including the image processing apparatus according to the present exemplary embodiment will be described with reference to the drawings. FIG. 1 illustrates a configuration of an image processing system 100 including an image processing apparatus 300 according to the present exemplary embodiment. The image processing apparatus 300 is connected to a tomographic image capturing apparatus (also referred to as an OCT) 200, an external storage unit 400, a display unit 500, and an input unit 600 via interfaces, of which the image processing system 100 is composed.

Further, the tomographic image capturing apparatus 200 is an apparatus that captures a tomographic image of an ocular portion. The apparatus used as the tomographic image capturing apparatus 200 includes, for example, an SS-OCT. The tomographic image capturing apparatus 200 is a known apparatus, and therefore will be described here omitting a detailed description thereof and focusing on settings of an image-capturing range where the tomographic image is captured and a parameter of an internal fixation lamp 204, which are set according to an instruction from the image processing apparatus 300.

Further, a galvanometer mirror 201 is used to scan the subject's eye with measurement light, and defines the image-capturing range where the subject's eye is imaged by the OCT. Further, a driving control unit 202 defines, in a planar direction of the subject's eye, the image-capturing range and the number of scan lines (a scan speed in the planar direction) by controlling a driving range and a speed of the galvanometer mirror 201. The galvanometer mirror 201 includes two mirrors, i.e., a mirror for X scan and a mirror for Y scan, and can scan a desired range of the subject's eye with the measurement light.

Further, the internal fixation lamp 204 includes a display unit 241 and a lens 242. A plurality of light-emitting diodes (LEDs) arranged in a matrix pattern is used as the display unit 241. A position where the light-emitting diode is lighted is changed according to a site desired to be imaged under control by the driving control unit 202. Light from the display unit 241 is guided to the subject's eye via the lens 242. The light emitted from the display unit 241 has a wavelength of 520 nm, and is displayed in a desired pattern by the driving control unit 202.

Further, a coherence gate stage 205 is controlled by the driving control unit 202 so as to deal with, for example, a difference in an axial length of the subject's eye. The coherence gate refers to a position where optical distances of the measurement light and reference light of the OCT match each other.

Further, the image processing apparatus 300 includes an image acquisition unit 301, a storage unit 302, an image processing unit 303, an instruction unit 304, and a display control unit 305. The image acquisition unit 301 is one example of the acquisition unit according to the aspect of the present invention. The image acquisition unit 301 includes a tomographic image generation unit 311. Then, the image acquisition unit 301 generates the tomographic image by acquiring signal data of the tomographic image captured by the tomographic image capturing apparatus 200 and performing signal processing thereon, and stores the generated tomographic image into the storage unit 302. The image processing unit 303 includes a registration unit 331 and an aqueous humor outflow pathway region acquisition unit 332. The aqueous humor outflow pathway region acquisition unit 332 is one example of the generation unit according to the aspect of the present invention, and includes a spatial differentiation processing unit 3321 and a projection processing unit 3322. The instruction unit 304 issues an instruction specifying the image-capturing parameters or the like to the tomographic image capturing apparatus 200.

Further, the external storage unit 400 holds information about the subject's eye (a name, an age, a gender, and the like of a patient), the captured image data, the image-capturing parameters, an image analysis parameter, and a parameter set by an operator in association with one another. The input unit 600 is, for example, a mouse, a keyboard, a touch operation screen, and/or the like, and the operator instructs the image processing apparatus 300 and the tomographic image capturing apparatus 200 via the input unit 600.

(Flow of Processing for Generating Image with Aqueous Humor Outflow Pathway Region Emphasized or Extracted Therein)

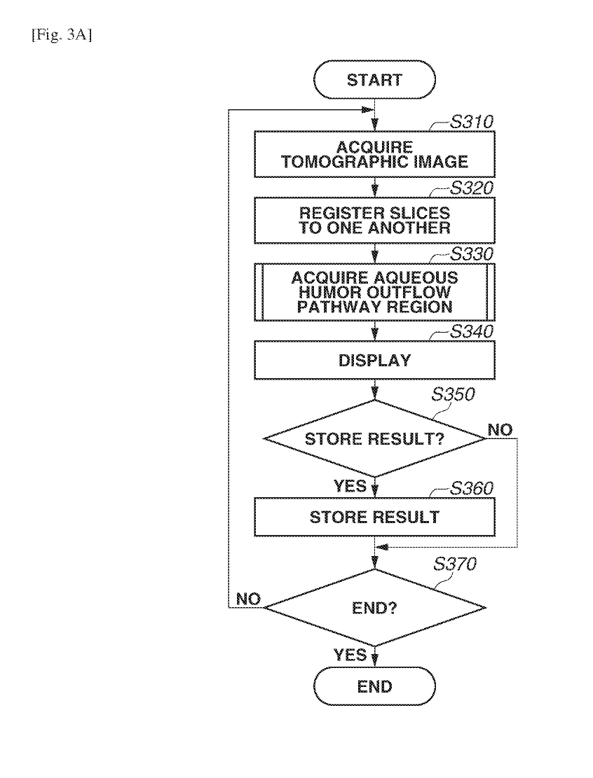

Next, a processing procedure performed by the image processing apparatus 300 according to the present exemplary embodiment will he described with reference to FIG. 3A. FIG. 3A is a flowchart illustrating a flow of processing in the entire present system according to the present exemplary embodiment.

(Step S310: Acquire Tomographic Image)

A subject's eye information acquisition unit (not illustrated) of the image, processing apparatus 300 acquires a subject identification number from the outside as information for identifying the subject's eye. The subjects eye information acquisition unit may be composed with use of the input unit 600. Then, the subject's eye information acquisition unit acquires the information about the subject's eye stored in the external storage unit 400 based on the subject identification number, and stores the acquired information into the storage unit 302.

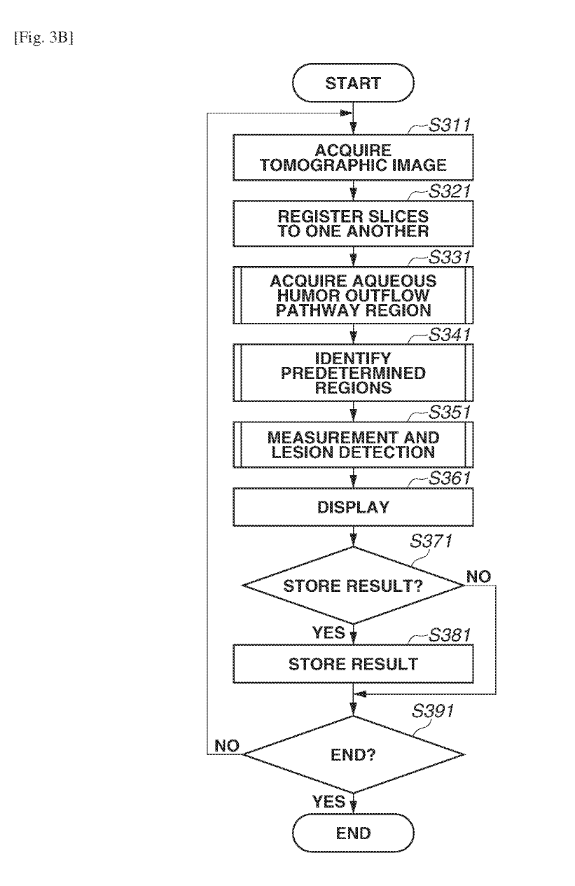

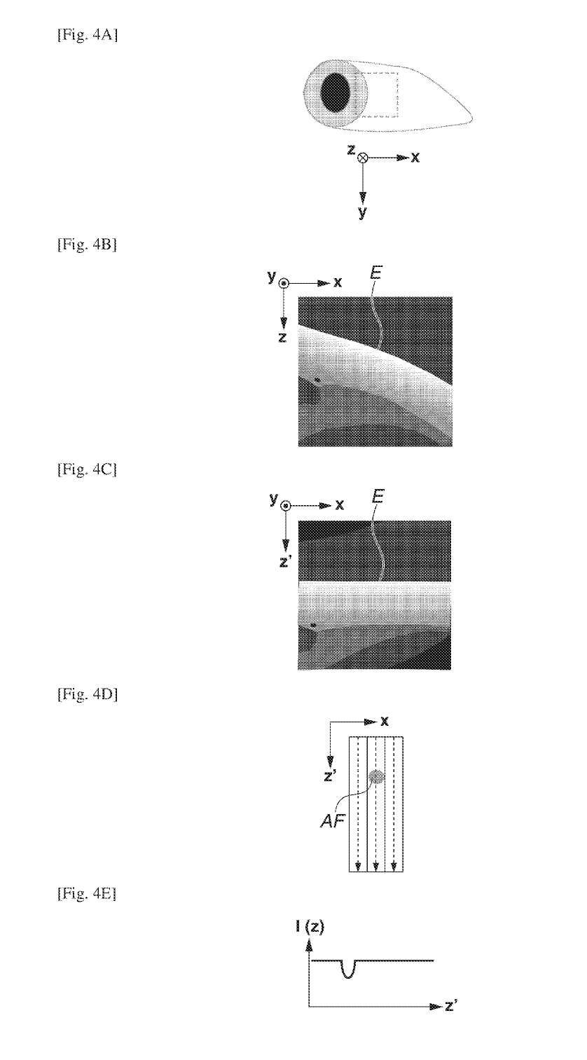

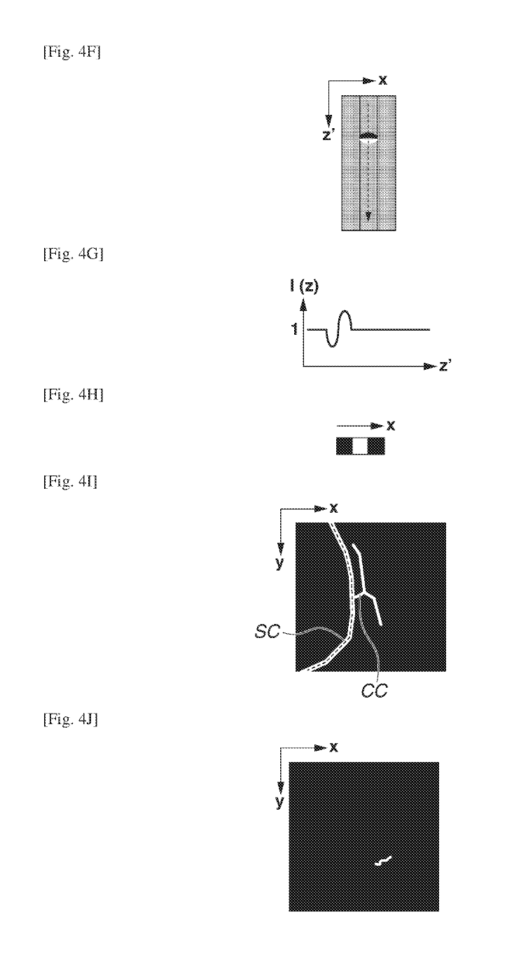

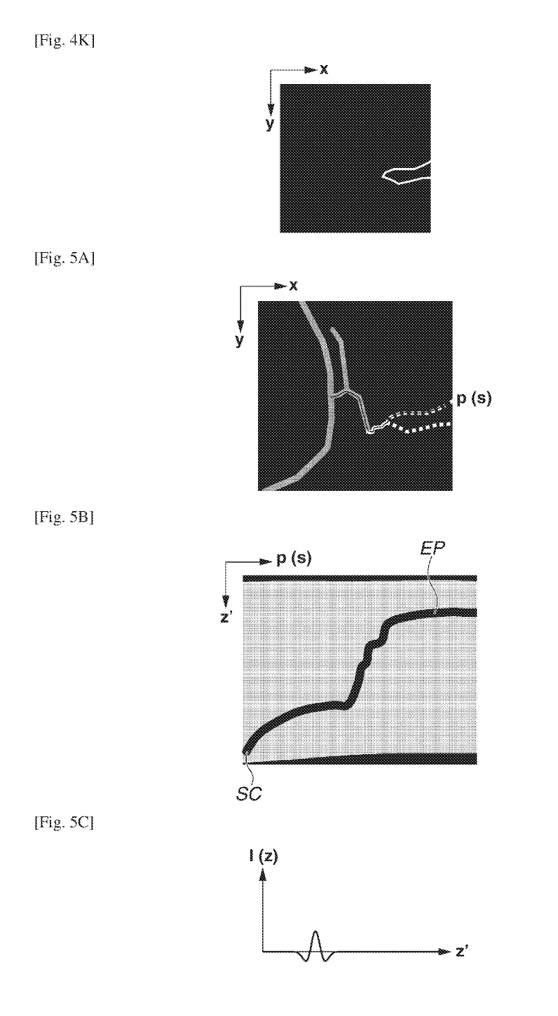

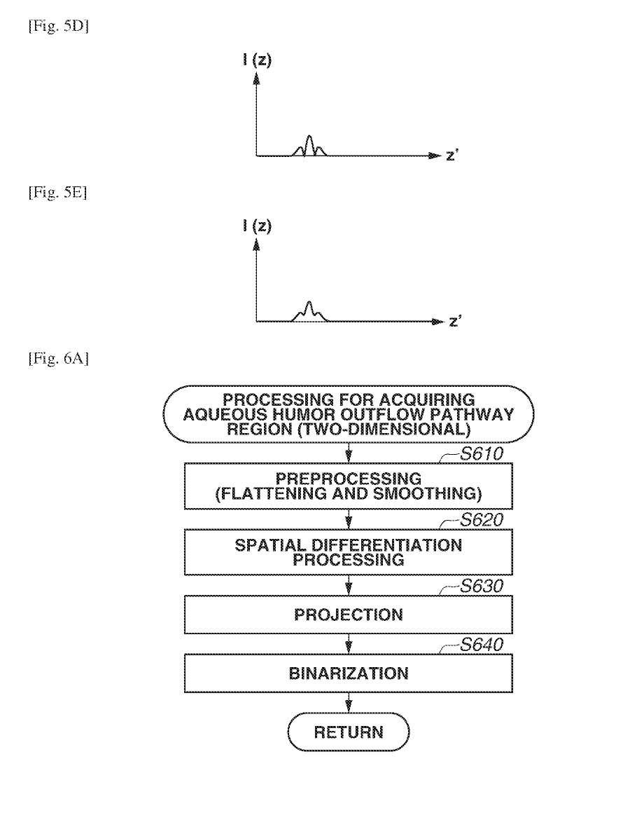

First, the tomographic image capturing apparatus 200 acquires the tomographic image according to the instruction from the instruction unit 304. The instruction unit 304 sets the image-capturing parameters, and the tomographic image capturing apparatus 200 captures the image according thereto. More specifically, the lightning position in the display unit 241 of the internal fixation lamp 204, the scan pattern of the measurement light that is defined by the galvanometer mirror 201, and the like are set. In the present exemplary embodiment, the driving control unit 202 sets the position of the internal fixation lamp 204 in such a manner that a junction between the cornea CN and the sclera S (for example, a scleral region indicated by a dotted line in FIG. 4A) in the anterior ocular segment is imaged, by controlling the light-emitting diodes of the display unit 241. A three-dimensional (3D) scan is employed as the scan pattern. Regarding the depth direction, scan positions are set in such a manner that the image-capturing range covers from the surface of the sclera S to the anterior chamber angle A. In the present exemplary embodiment, the image is captured once at each scan position, but the present invention also includes an embodiment in which the image is captured a plurality of times at each scan position. After these image-capturing parameters are set, the tomographic image of the subject's eye is captured. The tomographic image capturing apparatus 200 captures the tomographic image while causing the galvanometer mirror 201 to operate by controlling the driving control unit 202. As described above, the galvanometer mirror 201 includes the X scanner for a horizontal direction and the Y scanner for a vertical direction. Therefore, individually changing orientations of these scanners allows the subject's eye to be scanned in each of the horizontal direction (X) and the vertical direction (Y) in an apparatus coordinate system. Then, simultaneously changing the orientations of these scanners allows the subject's eye to be scanned in a direction that is a combination of the horizontal direction and the vertical direction, thereby allowing the subject's eye to be scanned in an arbitrary direction.

Then, the tomographic image generation unit 311 generates the tomographic image by acquiring the signal data of the tomographic image captured by the tomographic image capturing apparatus 200, and performing the signal processing thereon. In the present exemplary embodiment, the image processing system 100 will be described based on an example in which the SS-OCT is used as the tomographic image capturing apparatus 200. However, the tomographic image capturing apparatus 200 is not limited thereto, and the present invention also includes an embodiment in which the SD-OCT equipped with a light source having a long central wavelength (for example, 1 μm or longer) is used as the tomographic image capturing apparatus 200. First, the tomographic image generation unit 311 removes a fixed noise from the signal data. Next, the tomographic image generation unit 311 acquires data indicating an intensity with respect to the depth by carrying out spectral shaping and dispersion compensation and applying a discrete Fourier transform to this signal data. The tomographic image generation unit 311 generates the tomographic image by performing processing for cutting out an arbitrary region from the intensity data after the Fourier transform. The tomographic image acquired at this time is stored into the storage unit 302, and is also displayed on the display unit 500 in step S340, which will be described below.

(Step S320: Register Slices to One Another)

The registration unit 331 of the image processing apparatus 300 registers slices (two-dimensional tomographic images or B-scan images) in the three-dimensional tomographic image to one another. As a method for the registration, for example, an evaluation function expressing a degree of similarity between the images is defined in advance, and the image is deformed in such a manner that this evaluation function yields a highest value. Examples of the evaluation function include a method that makes the evaluation with use of a correlation coefficient. Further, examples of the processing for deforming the image include processing for translating or rotating the image with use of an affine transformation.

(Step S330: Processing for Acquiring (Processing for Emphasizing or Extracting) Aqueous Humor Outflow Pathway Region)

The aqueous humor outflow pathway region acquisition unit 332 generates the image in which the aqueous humor outflow pathway region extending from the Schlemm's canal region SC and including even the episcleral veins EP via the collector channel region CC is emphasized (drawn) with respect to the tomographic image registered in step S320. Further, the aqueous humor outflow pathway region acquisition unit 332 performs extraction processing by binarizing this emphasized image.

First, the image processing unit 303 performs flattening processing and smoothing processing with respect to the surface of the sclera S as preprocessing on the tomographic image registered in step S320. Next, the image processing unit 303 divides the tomographic image, on which the preprocessing has been performed, into a plurality of slice sections. The tomographic image can be divided into an arbitrary number of sections, but, in the present exemplary embodiment, a slice group corresponding to the scleral region is divided into three sections. Further, the image processing unit 303 generates a differential image by performing spatial differentiation processing on at least a deepest section among the divided sections. The image processing unit 303 generates the image in which the aqueous humor outflow pathway region including the Schlemm's canal region SC and the collector channel region CC is emphasized, by performing projection processing on this differential image based on the amount of the variation in the luminance value in the depth direction. The projection processing performed at this time is processing for generating a two-dimensional image acquired by projecting a value indicating a change in the luminance value acquired through the spatial differentiation processing on a plane intersecting with the depth direction. Further, the image processing unit 303 extracts the aqueous humor outflow pathway region including the Schlemm's canal region SC and the collector channel region CC by binarizing this highlighted image multi valued image). A specific content of the processing for acquiring (the processing for emphasizing or extracting) the aqueous humor outflow pathway region will be described in detail in descriptions of steps S610 to 5640.

(Step S340: Display)

The display control unit 305 displays, on the display unit 500, the tomographic image registered in step S320 and the projection image of the aqueous humor outflow pathway region that has been generated for each of the slice sections acquired by dividing the slice group into the three sections in step S330 (FIGS. 4I, 4J, and 4K). Further, the display control unit 305 respectively assigns a red (R) component, a green (G) component, and a blue (B) component, which are one example of a display manner, to the projection images (the two-dimensional images) of the aqueous humor outflow pathway region, each of which has been generated for each of the slice sections acquired by dividing the slice group into the three sections in step S330. Then, the display control unit 305 also displays, on the display unit 500, an image (FIG. 5A) acquired by combining into a color composite (displaying in a superimposed manner) the projection images of the aqueous humor outflow pathway region with the respective color components assigned thereto. The user can know the patency condition of the aqueous humor outflow pathway region in different depth ranges by observing this color composite image.

Further, the input unit 60 inputs a pathway p(s) (a portion indicated by a black solid line illustrated in FIG. 5A) extending from the aqueous humor outflow pathway region belonging to the deepest slice section (Red) and reaching the aqueous humor outflow pathway region in an intermediate layer (Green) and the aqueous humor outflow pathway region in an outermost layer (Blue) to this color composite image according to an instruction from the operator (the user). The display control unit 305 generates a curved planar image of the tomographic image along the pathway (FIG. 5B), thereby displaying this curved planar image including the aqueous humor outflow pathway region extending from the Schlemm's canal region SC and reaching the episcleral veins EP on the display unit 500. This curved planar image enables the user to know more easily the luminance value and a shape of the aqueous humor outflow pathway extending from the Schlemm's canal region SC and reaching the episcleral veins EP. The displayed image is not limited thereto, and, for example, the binary image of this projection image of the aqueous humor outflow pathway region may be displayed on the display unit 500.

(Step S350: Determine Whether Result Should be Stored)

The image processing apparatus 300 acquires, from the outside, an instruction specifying whether to store the tomographic image acquired in step S310, the image with the aqueous humor outflow pathway region emphasized therein and the binary image acquired in step S330, and the data displayed in step S340 into the external storage unit 400. This instruction is, for example, input by the operator via the input unit 600. If the image processing apparatus 300 is instructed to store them (YES in step S350), the processing proceeds to step S360. If the image processing apparatus 300 is not instructed to store them (NO in step S350), the processing proceeds to step S370.

(Step S360: Store Result)

The image processing unit 303 transmits an examination date and time, the information for identifying the subject's eye, and the storage target data determined in step S350 to the external storage unit 400 in association with one another.

(Step S370: Determine Whether to End Processing)

The image processing apparatus 300 acquires, from the outside, an instruction specifying whether to end the series of processes from steps S310 to S360. This instruction is input by the operator via the input unit 600. If the instruction to end the processing is acquired (YES in step S370), the processing is ended. On the other hand, if an instruction to continue the processing is acquired (NO in step S370), the processing returns to step S310, from which the processing is performed on a next subject's eye (or the processing is performed on the same subject's eye again).

(Processing for Acquiring (Processing for Emphasizing or Extracting) Aqueous Humor Outflow Pathway Region)

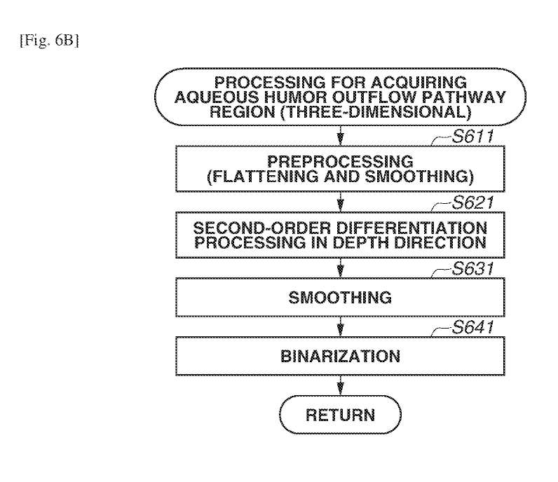

Further, details of the processing performed in step S330 will be described with reference to a flowchart illustrated in FIG. 6A.

(Step S610: Preprocessing (Flattening and Smoothing))

The image processing unit 303 performs the flattening processing and the smoothing processing with respect to the surface of the sclera S as the preprocessing on the tomographic image. FIG. 4B illustrates an example of the tomographic image of the anterior ocular segment before the flattening processing, and FIG. 4C illustrates an example of the tomographic image of the anterior ocular segment after the flattening processing. An arbitrary known method may be employed for the flattening processing performed at this time, but, in the present exemplary embodiment, the image processing unit 303 flattens the surface of the sclera S by detecting an edge E corresponding to the surface of the sclera S on each A-scan line in the tomographic image of the anterior ocular segment that is illustrated in FIG. 4B, and aligning adjacent A-scan lines with each other in the depth direction in such a manner that this edge E is located at a same depth position. This flattening processing is processing for facilitating an observation and image processing along a curved surface in parallel with the surface of the sclera S, and is not essential processing to the present invention. In a case where the flattening processing is omitted, the present processing for acquiring the aqueous humor outflow pathway region can be realized by performing processing of steps S620 to S640 on the tomographic image while referring to a pixel value belonging to the curved surface in parallel with the surface of the sclera S. Further, the image processing unit 303 performs the smoothing processing on the tomographic image that has been subjected to the flattening processing so as to reduce a noise. An arbitrary known smoothing method may be employed for the smoothing processing, but, in the present exemplary embodiment, the image processing unit 303 smooths the tomographic image with use of a Gaussian filter.

(Step S620: Spatial Differentiation Processing)

The image processing unit 303 divides the tomographic image of the anterior ocular segment, on which the flattening processing has been performed in step S610, into the plurality of slice sections. The number of sections into which the tomographic image of the anterior ocular segment is divided may be set to an arbitrary number, but, in the present exemplary embodiment, assume that the slice group substantially corresponding to the scleral region S is divided into the three sections at even intervals. The slice group substantially corresponding to the scleral region S can be determined by identifying low-luminance pixels (pixels each having a luminance value lower than a threshold value T1 and corresponding to an outside of an eyeball or to the angle region A) continuing from an end point of each A-scan line, and then determining the slice group substantially corresponding to the scleral region S as slices in which a proportion of these low-luminance pixels in each slice is smaller than a threshold value T2.

Further, the spatial differentiation processing unit 3321 performs the spatial differentiation processing on at least the tomographic image belonging to the deepest slice section among the slice sections acquired by dividing the tomographic image of the anterior ocular segment into the three sections by the image processing unit 303. In the present exemplary embodiment, assume that the spatial differentiation processing is performed on all the three slice sections. More specifically, the spatial differentiation processing unit 3321 performs the spatial differentiation processing by calculating division of the pixel value between the adjacent slices. In the present exemplary embodiment, the spatial differentiation processing will be described as calculating the division as one example thereof, but the present invention also includes an embodiment in which other spatial differentiation processing, such as subtraction processing, is performed.

As illustrated in FIG. 4D, the scleral region S exhibits such a characteristic that the luminance value thereof is evenly high but low only at the region AF belonging to the aqueous humor outflow pathway. FIGS. 4D and 4E illustrate a part of the tomographic image of the anterior ocular segment (a part corresponding to three A-scan lines) and a luminance profile on a central A-scan line illustrated in FIG. 4D, respectively. Therefore, as a result of the spatial differentiation, pixels in the scleral region S are grayed, and pixels in one and the other of boundaries of the aqueous humor outflow pathway region are blacked and whitened, respectively, as illustrated in FIG. 4F. FIG. 4F illustrates an example in a case where the luminance value in the lower slice (where a z′ coordinate value is large)/the luminance value in the upper slice (where the z′ coordinate value is small) is calculated as the spatial differentiation. The spatial differentiation is not limited thereto, and the present invention also includes an embodiment in which the luminance value in the upper slice (where the z′ coordinate value is small)/the luminance value in the lower slice (where the z′ coordinate value is large) is calculated as the spatial differentiation. Alternatively, the spatial differentiation may be carried out by the subtraction instead of the division between the luminance values of the adjacent slices. FIG. 4G illustrates a luminance profile on a central A-scan line illustrated in FIG. 4F. FIG. 4G makes it clear that the variation in the luminance value increases if the aqueous humor outflow pathway is contained in the A-scan line. However, in the case where the subtraction processing is performed as the differentiation processing, an offset in the luminance profile illustrated in FIG. 4G is placed at approximately zero (not approximately one).

(Step S630: Projection)

The projection processing unit 3322 emphasizes (draws) the deep aqueous humor outflow pathway region including the Schlemm's canal region SC and the collector channel region CC by carrying out the projection based on the amount of the variation in the luminance value (in the depth direction) of at least the spatial differential image corresponding to the deepest slice section among the spatial differential images generated in step S620. In the present exemplary embodiment, the projection processing unit 3322 calculates a standard deviation of the luminance value in an A-scan line direction at each pixel position in the spatial differential image corresponding to each of all the slice sections, i.e., the three slice sections, and generates a projection image having this standard deviation as a pixel value thereof. In the present exemplary embodiment, the standard deviation projection is carried out, but the projection is not limited thereto and an arbitrary known value may be calculated as long as this value is a value capable of quantifying a degree of the variation in the luminance value. For example, the present invention also includes an embodiment in which (a maximum value−a minimum value) is calculated or a variance is calculated instead of the standard deviation. In the spatial differential image corresponding to the three A-scan lines illustrated in FIG. 4F, carrying out the standard deviation projection in the depth direction results in an increase in this standard deviation at the pixel (the central pixel) including the aqueous humor outflow pathway region, and thus an increase in the luminance at this pixel (FIG. 4H) similarly to a result of contrast-enhanced imaging of this aqueous humor outflow pathway region.

In the present exemplary embodiment, assume that the projection images corresponding to the tomographic images of the deepest, intermediate, and outermost slice sections are generated as illustrated in FIGS. 4I, 4J, and 4K, respectively, by carrying out the standard deviation projection in the depth direction on the three spatial differential images generated in step S620.

(Step S640: Binarization)

The aqueous humor outflow pathway region acquisition unit 332 generates the binary image regarding the two-dimensional aqueous humor outflow pathway region by binarizing each of the projection images generated in step S630 based on the predetermined threshold value (processing for extracting the two-dimensional aqueous humor outflow pathway region). The method for the binarization is not limited thereto, and an arbitrary known binarization method may be employed.

According to the above-described configuration, the image processing apparatus 300 performs the processing for differentiating the luminance value in the depth direction on the tomographic image of the anterior ocular segment that contains at least the deep scleral portion. Next, the image processing apparatus 300 generates the image in which the two-dimensional aqueous humor outflow pathway region including the Schlemm's canal region SC and the collector channel region CC is emphasized or extracted, by carrying out the standard deviation projection in the depth direction with respect to the different depth ranges of this differential image, and binarizing the projection image. Due to this configuration, the image processing apparatus 300 can non-invasively emphasize or extract the two-dimensional aqueous humor outflow pathway region including the Schlemm's canal region SC and the collector channel region CC.

### Second Exemplary Embodiment

An image processing apparatus according to a second exemplary embodiment of the present invention will be described below. The image processing apparatus generates an image in which a three-dimensional aqueous humor outflow pathway region including the Schlemm's canal region SC and the collector channel region CC is emphasized (drawn), by calculating a second-order differential of the luminance value in the depth direction with respect to the tomographic image of the anterior ocular segment that contains at least the deep scleral portion, and calculating an absolute value of this second-order differential value. Further, the image processing apparatus performs the extraction processing by binarizing this emphasized image.

The image processing system 100 including the image processing apparatus 300 according to the present exemplary embodiment is configured in a similar manner to the configuration according to the first exemplary embodiment, and therefore a description thereof will be omitted below. Further, a flow of image processing according to the present exemplary embodiment is as illustrated in FIG. 3A, and steps except for steps S330 and 5340 are similar to the steps according to the first exemplary embodiment and therefore descriptions thereof will be omitted below.

(Step S330: Processing for Acquiring (Processing for Emphasizing or Extracting) Aqueous Humor Outflow Pathway Region)

The aqueous humor outflow pathway region acquisition unit 332 generates the image in which the three-dimensional aqueous humor outflow pathway region extending from the Schlemm's canal region SC and also including even the episcleral veins EP via the collector channel region CC is emphasized (drawn), with use of the tomographic image of the anterior ocular segment that has been registered in step S320. Further, the aqueous humor outflow pathway region acquisition unit 332 extracts the three-dimensional aqueous humor outflow pathway region by binarizing this emphasized image based on a predetermined threshold value.

First, the image processing unit 303 performs the flattening processing and the smoothing processing with respect to the surface of the sclera S as the preprocessing on the three-dimensional tomographic image of the anterior ocular segment that has been registered among the slices in step S320. Next, the spatial differentiation processing unit 3321 generates the image (the three-dimensional image) in which the three-dimensional aqueous humor outflow pathway region including the Schlemm's canal region SC and the collector channel region CC is emphasized (drawn), by performing the processing for calculating the second-order differential of the luminance value in the depth direction on the tomographic image of the anterior ocular segment that has been subjected to the preprocessing, calculating the absolute value of this second-order differential value, and smoothing the differential image. The method described in the present step is less affected by an influence of a reduction in the luminance value in the tomographic image according to deepening of the position in the sclera S, and therefore can generate an image in which the aqueous humor outflow pathway region in the deep scleral portion is highly contrastively emphasized compared to displaying the aqueous humor outflow pathway region at a high luminance by simply inverting the luminance value. Further, the aqueous humor outflow pathway region acquisition unit 332 extracts the three-dimensional aqueous humor outflow pathway region by binarizing this image (the multivalued image) with the three-dimensional aqueous humor outflow pathway region emphasized therein based on the predetermined threshold value. A specific content of the processing for acquiring (the processing for emphasizing or extracting) the aqueous humor outflow pathway region will be described in detail in descriptions of steps S611 to S641.

(Step S340: Display)

The display control unit 305 displays, on the display unit 500, the three-dimensional tomographic image registered among the slices in step S320, and the multivalued image with the three-dimensional aqueous humor outflow pathway region emphasized (drawn) therein that has been generated in step S330. The displayed image is not limited thereto, and, for example, the display control unit 305 may display, on the display unit 500, the binary image regarding the three-dimensional aqueous humor outflow pathway region that has been generated by binarizing this multivalued image based on the predetermined threshold value. At this time, the display control unit 305 can display a plurality of two-dimensional images at different positions in the depth direction that forms the generated three-dimensional image, continuously along the depth direction on the display unit 500 (as a moving image). This display enables the user to more easily three-dimensionally know the, pathway p(s) of the aqueous humor outflow pathway region. Besides the method that continuously displays the moving image as described above, the display control unit 305 may be configured to (three-dimensionally) display the three-dimensional image with the aqueous humor outflow pathway region emphasized (drawn) therein by volume rendering on the display unit 500. Further, details of the processing performed in step S330 will he described with reference to a flowchart illustrated in FIG. 6B. Step S611 is similar to the processing in step S610 according to the first exemplary embodiment, and therefore a description thereof will be omitted here.

(Step S621: Second-Order Differentiation Processing in Depth Direction)

The spatial differentiation processing unit 3321 performs the processing for calculating the second-order differential of the luminance value in the depth direction on the three-dimensional tomographic image of the anterior ocular segment that has been subjected to the flattening processing in step S611. For example, if the tomographic image of the anterior ocular segment that has been already subjected to the flattening processing exhibits the luminance profile illustrated in FIG. 4E, the processing for calculating the second-order differential of the luminance value between the adjacent slices leads to a luminance profile like an example illustrated in FIG. 5C. In the present exemplary embodiment, the spatial differentiation processing unit 3321 performs the processing for subtracting the luminance value between the adjacent slices (the luminance value in the lower slice (where the z′ coordinate value is large)−the luminance value in the upper slice (where the z′ coordinate value is small)) as the differential processing. The differentiation processing is not limited thereto, and, for example, the spatial differentiation processing unit 3321 may perform the processing for calculating the division of the luminance value between the adjacent slices. However, in the case of the division processing, an offset in the luminance profile illustrated in FIG. 5C is placed at approximately one (not approximately zero). Further, the calculation may he made with a first term and a second term interchanged with each other in the subtraction processing or a denominator and a numerator interchanged with each other in the division processing. Alternatively, the subtraction processing may be performed after this division processing or the division processing may be performed after the subtraction processing, and the present invention also includes such an embodiment. However, in the case where the division processing is performed after the subtraction processing, the division processing should be performed after a predetermined positive value is added to a value resulting from the subtraction (so that this value resulting from the subtraction becomes a positive value).

Next, the pixels on each A-scan line in the acquired second-order differential image include both a larger value and a smaller value than the offset value (approximately zero in the case where the subtraction processing is performed, or approximately one in the case where the division processing is performed). Therefore, when the same aqueous humor outflow pathway region contained in this second-order differential image is observed while the slice number is changed, the luminance value is inverted in the middle of the observation (the aqueous humor outflow pathway region is observed as a black region first, is changed into a white region next, and then returns to the black region lastly). In the present exemplary embodiment, the absolute value of the value acquired by calculating the second-order differential of the luminance value is calculated (FIG. 5D) to avoid such inversion of the luminance value. The processing for avoiding the inversion of the luminance value is not limited to the processing for calculating the absolute value (of the second-order differential value), and may be realized by, for example, performing processing for setting the pixel value to zero at such a pixel that the second-order differential value indicates a negative value in FIG. 5C. Further, assume that, in the case where the processing for further calculating the division of the luminance value between the adjacent slices is performed on the image after the division processing as the second-order differentiation, the absolute value is calculated after one is subtracted from this second-order differential value so as to yield a luminance profile similar to the luminance profile illustrated in FIG. 5D.

(Step S631: Smoothing)

The aqueous humor outflow pathway region acquisition unit 332 performs the smoothing processing on the differential image acquired in step S621 (the image acquired by calculating the second-order differential of the luminance value and calculating the absolute value thereof) to improve continuity of the luminance value in the same aqueous humor outflow pathway region and reduce a background noise. Arbitrary smoothing processing can be employed, but, in the present exemplary embodiment, the differential image is smoothed with use of the Gaussian filter. The luminance profile (FIG. 5D) formed by the processing in step S621 is changed into a luminance profile like an example illustrated in FIG. 5E by the processing in the present step. Further, the image formed by the processing in the present step will be referred to as the image with the three-dimensional aqueous humor outflow pathway region emphasized (drawn) therein.

(Step S641: Binarization)

The aqueous humor outflow pathway region acquisition unit 332 generates the binary image regarding the three-dimensional aqueous humor outflow pathway region (performs the processing for extracting the three-dimensional aqueous humor outflow pathway region) by binarizing the image with the three-dimensional aqueous humor outflow pathway region emphasized therein (that has been generated in step S631) based on the predetermined threshold value. The method for the binarization is not limited thereto, and an arbitrary known binarization method may be employed. For example, the image may be binarized based on a different threshold value for each local region instead of being binarized based on the single threshold value. Alternatively, the three-dimensional aqueous humor outflow pathway region may be further correctly extracted by the following procedure. For example, edge-preserving smoothing processing is performed on the three-dimensional tomographic image of the anterior ocular segment (that has been already subjected to the flattening processing) in advance. Next, thinning processing is performed after the emphasized image acquired in step S631 (or the second-order differential image) is binarized based on a predetermined threshold value. The three-dimensional aqueous humor outflow pathway region may be extracted by performing three-dimensional region growing processing on the tomographic image that has been already subjected to the edge-preserving smoothing processing while setting a pixel group (connected components) acquired from this thinning processing as a seed point (a starting point).

In the present exemplary embodiment, the processing for acquiring the aqueous humor outflow pathway region has been described based on the example that generates the image with the three-dimensional aqueous humor outflow pathway region emphasized or extracted therein based on the value acquired by calculating the second-order differential of the luminance value in the depth direction in the three-dimensional tomographic image of the anterior ocular segment on which the flattening processing has been performed. However, the present invention is not limited only thereto. For example, not only the aqueous humor outflow pathway region acquisition unit 332 generates the image with the three-dimensional aqueous humor outflow pathway region emphasized therein, but also the projection processing unit 3322 may generate an image with the two-dimensional aqueous humor outflow pathway region emphasized therein by projecting this image with the three-dimensional aqueous humor outflow pathway region emphasized therein (or the second-order differential image generated in step S621). Further, the aqueous humor outflow pathway region acquisition unit 332 may extract the two-dimensional aqueous humor outflow pathway region by binarizing this image with the two-dimensional aqueous humor outflow pathway region emphasized therein based on a predetermined threshold value. Alternatively, the projection processing unit 3322 may generate an image projected in a limited projection range with the two-dimensional aqueous humor outflow pathway region emphasized therein by projecting a partial image of the three-dimensional image of the aqueous humor outflow pathway region (or a partial image of the second-order differential image generated in step S621), and the present invention also includes such an embodiment. Further, the aqueous humor outflow pathway region acquisition unit 332 may perform the extraction processing by binarizing this image projected in the limited projection range with the two-dimensional aqueous humor outflow pathway region emphasized therein based on a predetermined threshold value, and the present invention also includes such an embodiment. An arbitrary known projection method, such as the standard deviation projection and average intensity projection, may be employed as the method for the projection in the case where the projection is carried out. In the case where the entire image with the three-dimensional aqueous humor outflow pathway region emphasized therein or the entire second-order differential image generated in step S621 is projected, it is desirable to carry out the projection by carrying out maximum intensity projection or calculating (the maximum value−the minimum value) of the luminance value for each A-scan line to increase a contrast in the projected image. The direction for the projection is not limited to the depth direction, and the projection may be carried out in an arbitrary direction. However, in the case where the differential image is used, it is desirable that the direction for the differentiation and the direction for the projection substantially coincide with each other (to increase the contrast in the projected image as much as possible).

Further, the image displayed on the display unit 500 is not limited to the image with the three-dimensional aqueous humor outflow pathway region emphasized therein and the binary image of this emphasized image. For example, the image with the two-dimensional aqueous humor outflow pathway region emphasized therein that is generated by projecting the image with the three-dimensional aqueous humor outflow pathway region emphasized therein (or the second-order differential image generated in step S621) may also be displayed on the display unit 500. Alternatively, after the projection processing unit 3322 generates the image projected in the limited projection range with the two-dimensional aqueous humor outflow pathway region emphasized therein by projecting the partial image of the three-dimensional image of the aqueous humor outflow pathway region (or the partial image of the second-order differential image generated in step S621), this generated image may be displayed on the display unit 500, and the present invention also includes such an embodiment. Alternatively, similarly to the display in the first exemplary embodiment, different display manners may be assigned to a group of images with the two-dimensional aqueous humor outflow pathway region emphasized therein that is generated by projecting the image with the three-dimensional aqueous humor outflow pathway region emphasized therein (or the second-order differential image generated in step S621) with respect to different depth ranges, and this group of images may be displayed in a superimposed manner. Further, the binary image of the image with the two-dimensional aqueous humor outflow pathway region emphasized therein, the binary image of the image projected in the limited projection range with the two-dimensional aqueous humor outflow pathway region emphasized therein, and an image acquired by binarizing this superimposed image, each of which is generated from the binarization based on the predetermined threshold value, may be displayed on the display unit 500.

According to the above-described configuration, the image processing apparatus 300 performs the following processing. Specifically, the image processing apparatus 300 generates the image in which the three-dimensional aqueous humor outflow pathway region including the Schlemm's canal region SC and the collector channel region CC is emphasized or extracted, by calculating the second-order differential of the luminance value in the depth direction with respect to the tomographic image of the anterior ocular segment that contains at least the deep scleral portion, calculating the absolute value of this second-order differential value, smoothing the differential image, and binarizing the smoothed image. By this processing, the image processing apparatus 300 can non-invasively emphasize or extract the three-dimensional aqueous humor outflow pathway region including the Schlemm's canal region SC and the collector channel region CC.

### Third Exemplary Embodiment

An image processing apparatus according to a third exemplary embodiment of the present invention will be described below. The image processing apparatus identifies the Schlemm's canal region SC and the collector channel region CC from the aqueous humor outflow pathway region including the Schlemm's canal region SC and the collector channel region CC extracted with use of a similar image processing method to the second exemplary embodiment, and measures a diameter or a cross-sectional area of this aqueous humor outflow pathway region. Further, the image processing apparatus detects a lesion candidate region, such as stenosis, based on a statistical value of this measured value.

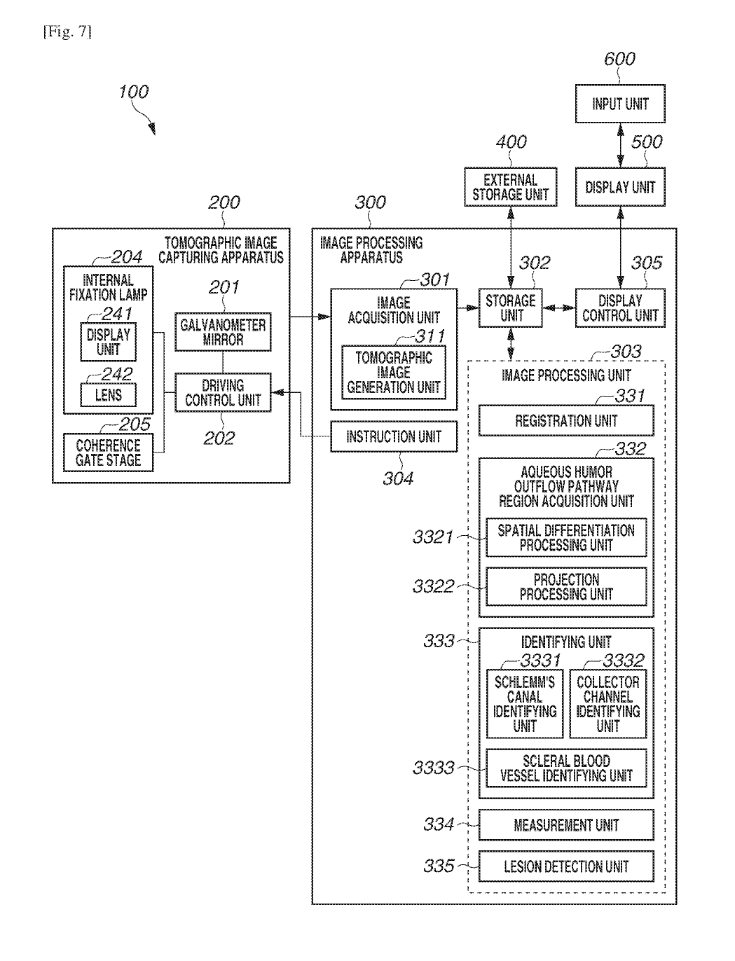

FIG. 7 illustrates a configuration of the image processing system 100 including the image processing apparatus 300 according to the present exemplary embodiment. The third exemplary embodiment is different from the second exemplary embodiment in terms of the image processing unit 303 including an identifying unit 333, a measurement unit 334, and a lesion detection unit 335. The identifying unit 333 includes a Schlemm's canal identifying unit 3331, a collector channel identifying unit 3332, and a scleral blood vessel identifying unit 3333. Further, the Schlemm's canal identifying unit 3331, the collector channel identifying unit 3332, and the scleral blood vessel identifying unit 3333 are one example of an identifying unit according to one aspect of the present invention. Further, a flow of image processing according to the present exemplary embodiment is as illustrated in FIG. 3B, and steps except for steps S341, S351, and S361 are similar to the steps according to the second exemplary embodiment and therefore descriptions thereof will be omitted below.

(Step S341: Identify Predetermined Regions)

The identifying unit 333 identifies the Schlemm's canal region SC, the collector channel region CC, and the scleral blood vessel region with respect to the three-dimensional aqueous humor outflow pathway region extracted in step S331 based on an anatomical characteristic of the aqueous humor outflow pathway. A specific content of processing for identifying the Schlemm's canal region SC, the collector channel region CC, and the scleral blood vessel region will be described in detail in descriptions of steps S810 to S840.

(Step S351: Measurement and Lesion Detection)

The measurement unit 334 measures the diameter or the cross-sectional area as the measured value regarding the aqueous humor outflow pathway region extracted in step S331. Further, the lesion detection unit 335 compares this measured value with values in a predetermined normal value range, and detects the aqueous humor outflow pathway region having the measured value outside this normal value range as the lesion candidate region. A specific content of the measurement and lesion detection processing will be described in detail in descriptions of steps S850, S855, and S860.

(Step S361: Display)

The display control unit 305 displays the images displayed in the second exemplary embodiment (the registered tomographic image, the image with the three-dimensional aqueous humor outflow pathway region emphasized therein, and the binary image of this emphasized image) on the display unit 500. Further, the display control unit 305 presents the display with a predetermined display manner (for example, a predetermined color) assigned to the Schlemm's canal region SC and the collector channel region CC identified in step S341, and/or displays a distribution regarding the measured value and the lesion candidate region (for example, stenosis) acquired in step S351 on the display unit 500.

(Flow of Processing for Identifying Predetermined Regions)

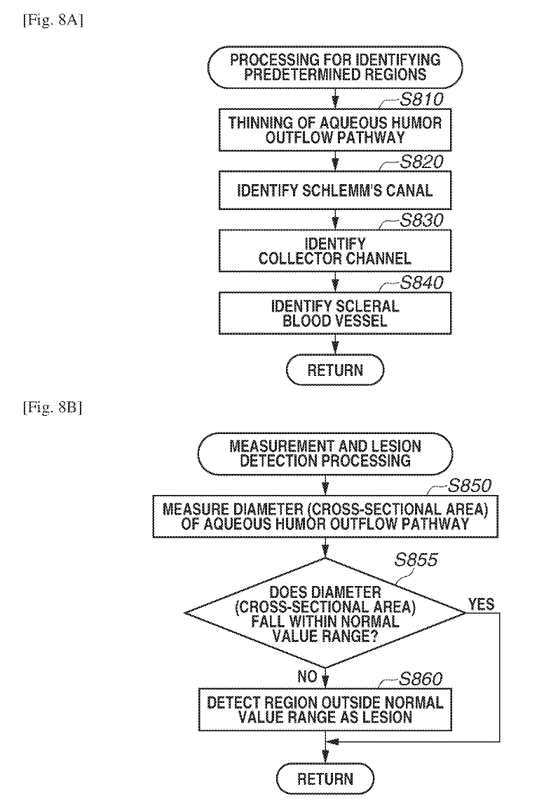

Further, details of the processing performed in step S341 will be described with reference to a flowchart illustrated in FIG. 8A.

(Step S810: Thinning of Aqueous Humor Outflow Pathway Region)

The identifying unit 333 performs three-dimensional thinning processing on the aqueous humor outflow pathway region extracted in step S331. Further, the identifying unit 333 labels a pixel group branch by branch by classifying the pixel group (connected components) acquired from the thinning processing into i) an end point (or an isolated point), ii) an internal point in a branch, and iii) a branch point based on the number of connections, and assigning a same label (a pixel value) to the pixel group from an end point or a branch point to a branch point or an end point adjacent thereto.

(Step S820: Identify Schlemm's Canal)

The Schlemm's canal identifying unit 3331 identifies the Schlemm's canal region SC based on the binary image of the three-dimensional aqueous humor outflow pathway region that has been generated in step S641. In the treatment of glaucoma that aims at the recovery of the flow amount of the aqueous humor passing through the Schlemm's canal region SC, such as the aqueous humor outflow pathway reconstruction surgery, to figure out the patency (no occurrence of stenosis or occlusion) of the Schlemm's canal region SC and the collector channel region CC adjacent thereto is important in determining the treatment position that can be expected to ensure the reduction in the intraocular pressure. Therefore, in the present exemplary embodiment, the Schlemm's canal region SC is identified in the present step, and the region corresponding to the collector channel region CC is identified in the next step.

In the present exemplary embodiment, the Schlemm's canal identifying unit 3331 identifies a pixel group that meets the following conditions in the three-dimensional aqueous humor outflow pathway region extracted in step S641 as the Schlemm's canal region SC. More specifically, the Schlemm's canal identifying unit 3331 identifies, as the Schlemm's canal region SC, the three-dimensional aqueous humor outflow pathway region belonging to a predetermined depth range and including a pathway (a branch) located on a closest side to a corneal center from the pathway (branch) groups labeled in step S810. The method for the binarization is not limited to the processing based on the threshold value, and an arbitrary known binarization method may be employed. In the present exemplary embodiment, assume that the predetermined depth range is set to a same depth range as the deepest slice section among the slice sections acquired by dividing the tomographic image into the three sections by a similar method to step S620 in the first exemplary embodiment. Further, information about on which side the corneal center is located with respect to the image is determined based on a visual fixation position.

(Step S830: Identify Collector Channel)

The collector channel identifying unit 3332 identifies the collector channel region CC based on the Schlemm's canal region SC identified in step S820. In the present exemplary embodiment, the collector channel identifying unit 3332 identifies a pixel group that meets the following conditions in the three-dimensional aqueous humor outflow pathway region extracted in step S641 as the collector channel region CC. More specifically, the collector channel identifying unit 3332 identifies, as the collector channel region CC, the three-dimensional aqueous humor outflow pathway region connected to the branch included in the Schlemm's canal region SC identified in step S820 and including a branch running toward a distal side (in a substantially opposite direction from the corneal central side) (among the branches labeled in step S810).

(Step S840: Identify Scleral Blood Vessel Region)

The scleral blood vessel identifying unit 3333 identifies the scleral blood vessel region as a region that excludes the Schlemm's canal region SC and the collector channel region CC identified in steps S820 and S830. In the present exemplary embodiment, the scleral blood vessel identifying unit 3333 identifies a pixel group that meets the following conditions in the three-dimensional aqueous humor outflow pathway region extracted in step S641 as the scleral blood vessel region. First, the scleral blood vessel identifying unit 3333 identifies a branch group that excludes the branches included in the Schlemm's canal region SC and the collector channel region CC identified in steps S820 and 830, from the branch groups labeled in step S810. Further, the scleral blood vessel identifying unit 3333 identifies the three-dimensional aqueous humor outflow pathway region that includes this branch group (excluding the branches included in the Schlemm's canal region SC and the collector channel region CC) as the scleral blood vessel region.

(Flow of Measurement and Lesion Detection Processing)

Further, details of the processing performed in step S351 will he described with reference to a flowchart illustrated in FIG. 8B.

(Step S850: Measure Diameter (Cross-Sectional Area) of Aqueous Humor Outflow Pathway Region)

The measurement unit 334 measures a diameter or a cross-sectional area of the Schlemm's canal region SC identified in step S820, the collector channel region CC identified in step S830, or the scleral blood vessel region identified in step S840 for each of the pathways (the branches) labeled in step S810. More specifically, the measurement unit 334 measures the diameter or the cross-sectional area of the aqueous humor outflow pathway region in a direction perpendicular to this branch at predetermined intervals along this branch.

(Step S855: Determine Whether Measured Diameter (Cross-Sectional Area) Falls within Normal Value Range)

The lesion detection unit 335 compares the measured value (the diameter or the cross-sectional area) regarding the aqueous humor outflow pathway region that has been measured in step S850 with the values in the normal value range set for this measured value. If the measured value falls outside the normal value range (NO in step S855), the processing proceeds to step S860. If the measured value falls within the normal value range (YES in step S855), the processing in the present step is ended.

(Step S860: Detect Region Outside Normal Value Range as Lesion)

The lesion detection unit 335 detects, as the lesion candidate region, a region in which the measured value has fallen outside the normal value range in the comparison processing in step S855. In the present exemplary embodiment, the lesion detection unit 335 detects a region having a smaller measured value than this normal value range as a stenosis portion. More specifically, the lesion detection unit 335 determines the stenosis portion if the measured value (the diameter or the cross-sectional area) regarding the Schlemm's canal region SC, the collector channel region CC, or the scleral blood vessel region that has been measured in step S850 is smaller than the normal value range and larger than a predetermined micro value Ts, while detecting this region as an occlusion portion if the measured value is smaller than this predetermined micro value Ts.

The method for detecting the lesion candidate region is not limited to the method based on the comparison with the values in the normal value range, and an arbitrary known method for detecting the lesion may be employed. For example, the present invention also includes the following embodiment. The measured value and the statistical value (for example, an average value or a median value) of this measured value are calculated for each of the branches in the aqueous humor outflow pathway region. Next, a ratio of each measured value to this statistical value is calculated, and the stenosis portion or the occlusion portion is detected based on this ratio.

Further, in the present exemplary embodiment, the image processing apparatus 300 has been described based on the example that identifies the Schlemm's canal region SC, the collector channel region CC, and the scleral blood vessel region with respect to the three-dimensional aqueous humor outflow pathway region extracted in step S641, and measures the three-dimensional shape to detect the lesion based on this measured value, but the present invention is not limited thereto. For example, the processing for identifying the Schlemm's canal region SC, the collector channel region CC, and the scleral blood vessel, the measurement processing, and the lesion detection processing may be performed on the binary image of the two-dimensional aqueous humor outflow pathway region (projected within the limited projection range) that is generated based on the method described in the description of the first exemplary embodiment or around the end of the description of the second exemplary embodiment. Alternatively, the processing for identifying the Schlemm's canal region SC, the collector channel region CC, and the scleral blood vessel, the measurement processing, and the lesion detection processing may be performed on the binary image of the two-dimensional aqueous humor outflow pathway region that is generated based on the method described around the end of the description of the second exemplary embodiment.

Further, the processing for identifying the two-dimensional Schlemm's canal can be performed by the following procedure. More specifically, the identifying unit 333 acquires the pathway (branch) group by performing the thinning processing on the binary image of the projection image (FIG. 4I) corresponding to the deepest slice section set by the image processing unit 303 according to a similar procedure to step S620 in the first exemplary embodiment, and labeling the pixel group. Further, the Schlemm's canal identifying unit 3331 identifies, as the Schlemm's canal region SC, the two-dimensional aqueous humor outflow pathway region including two branches (a portion indicated by a black dotted line in FIG. 4I) belonging to the closest side to the corneal center at each y coordinate from this branch group. Assume that the information about on which side the corneal center is located with respect to the image is determined based on the visual fixation position. In the example illustrated in FIG. 4I, the left side corresponds to the corneal central side.

Further, in the processing for identifying the two-dimensional collector channel region CC, the collector channel identifying unit 3332 identifies, as the collector channel region CC, the two-dimensional aqueous humor outflow pathway region connected to the (two) branches included in the Schlemm's canal region SC and including the branch running toward the distal side from this branch group.

Further, the processing for identifying the two-dimensional scleral blood vessel region is performed by a similar method to the processing for identifying the three-dimensional scleral blood vessel region. More specifically, the scleral blood vessel identifying unit 3333 excludes the branches included in the Schlemm's canal region SC and the collector channel region CC from the branch group (labeled by the identifying unit 333). The scleral blood vessel identifying unit 3333 identifies, as the scleral blood vessel region, the two-dimensional aqueous humor outflow pathway region including this branch group that excludes the branches included in the Schlemm's canal region SC and the collector channel region CC.

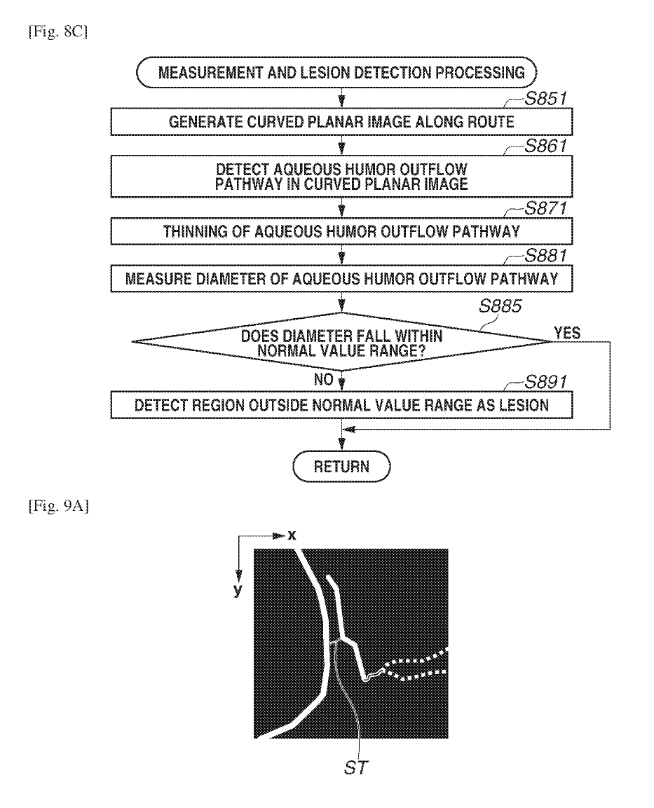

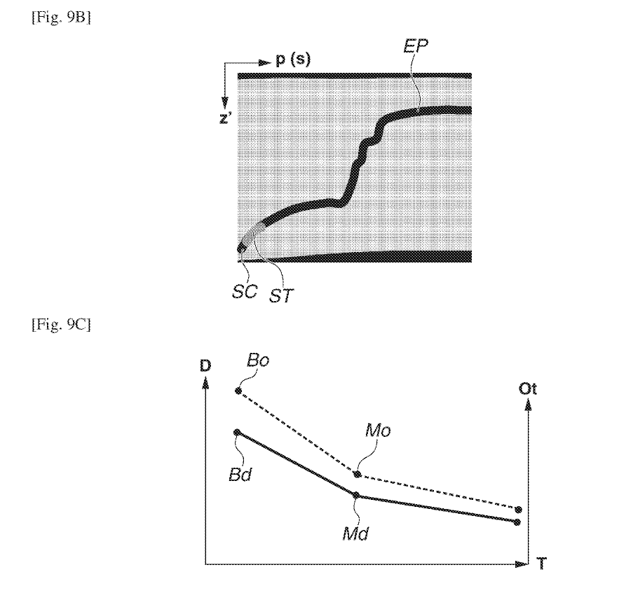

Further, in the two-dimensional measurement processing, the measurement unit 3334 measures the diameter of the aqueous humor outflow pathway region at predetermined intervals along the pathway (the branch) that the identifying unit 333 has acquired by performing the thinning processing and labeling the pixel group with respect to a group of binary images of projection images. However, the “group of binary images of projection images” refers to any of i) the group of binary images of the two-dimensional aqueous humor outflow pathway region that is generated based on the method described around the end of the description of the second exemplary embodiment, and ii) the group of binary images of the two-dimensional aqueous humor outflow pathway region (projected within the limited projection range) that is generated based on the method described in the description of the first exemplary embodiment or around the end of the description of the second exemplary embodiment. As the two-dimensional lesion detection processing, the measured value acquired from the two-dimensional measurement processing is compared with the values in the normal value range. If the measured value falls outside this normal value range, a region having this measured value is detected as the lesion candidate region. The method for detecting the lesion is not limited to the comparison with the normal value range, similarly to the method for the three-dimensional lesion detection processing. For example, the measured value and the statistical value (for example, the average value or the median value) of this measured value may be calculated in advance for each of the branches in the aqueous humor outflow pathway region, and the stenosis portion or the occlusion portion may be detected based on the ratio between this measured value and this statistical value. FIG. 9A illustrates an example of a map in which the lesion candidate region (a stenosis portion ST; a gray portion) detected in step S351 is superimposed on the image with the two-dimensional aqueous humor outflow pathway region emphasized therein. This example indicates that the shape of the venous region in the sclera S is normal, but stenosis occurs in the collector channel region CC.

Further, the two-dimensional measurement and lesion detection are not limited to the above-described processing performed on the binary images of the projection images. For example, the present invention also includes an embodiment in which the two-dimensional measurement and lesion detection are carried out on a curved planar image of the three-dimensional tomographic image of the anterior ocular segment that is generated along the pathway set on these projection images (or the binary images of these projection images) based on a processing flow like an example illustrated in FIG. 8C. For example, the image processing apparatus 300 may detect a region having a value lower than a predetermined value in this curved planar image as the aqueous humor outflow pathway region, and measure the shape (the diameter or the like) of the aqueous humor outflow pathway region in this curved planar image, thereby displaying the distribution of this measured value and/or detecting the lesion based on this two-dimensional shape value to then display the distribution of this lesion candidate region. The lesion detection unit 335 detects, as the lesion candidate region, a region in which the measured value (for example, the diameter) of the aqueous humor outflow pathway region measured in this curved planar image is smaller than the predetermined value. FIG. 9B illustrates an example in a case where the lesion candidate region detected in step S891 is displayed in a manner superimposed on this curved planar image. This example makes it clear that the veins in the sclera S are normal but stenosis occurs in the collector channel region CC.

According to the above-described configuration, the image processing apparatus 300 identifies the Schlemm's canal region SC, the collector channel region CC, and the scleral blood vessel region with respect to the aqueous humor outflow pathway region including the Schlemm's canal region SC and the collector channel region CC that is extracted by the similar image processing to the second exemplary embodiment, and measures the diameter or the cross-sectional area of this aqueous humor outflow pathway region. Further, the image processing apparatus 300 detects the lesion candidate region (stenosis or the like) based on this measured value. This processing enables the user to know whether there is stenosis or occlusion in the aqueous humor outflow pathway including the Schlemm's canal region SC and the collector channel region CC.

R. Poddar et al. (“In vivo volumetric depth-resolved vasculature imaging of human limbus and sclera with 1 μm swept source phase-variance optical coherence angiography”, J. Opt. 17 (6), June 2015) discusses a technique that draws (highlights) the veins in the front layer of the sclera by imaging a variance (a phase variance) of a phase shift amount of an OCT signal at the time of the imaging of the sclera with use of the SS-OCT. In this non-patent literature, a same position should be scanned three times, whereby the scan speed is increased compared to a technique that scans each position once, so as to reduce a time period from a start to an end of the scan as much as possible. Therefore, in this non-patent literature, the tomographic image is captured at a low resolution. Further, in this non-patent literature, because the OCT signal is attenuated on the deep layer side of the sclera, the phase-variance method cannot achieve the drawing (the highlighted display) or the extraction of the Schlemm's canal, the collector channel, and the deep scleral venous plexus.

### Other Exemplary Embodiments

In the above-described exemplary embodiments, the image processing apparatus 300 has been described based on the example that stores, into the external storage unit 400, the tomographic image captured in the same examination, and the image with the aqueous humor outflow pathway region emphasized therein and the binary image that are generated based on this tomographic image. However, the present invention is not limited thereto. For example, the present invention also includes an embodiment in which the image processing apparatus 300 stores each of tomographic images captured at different examination dates and times, the measured value regarding the aqueous humor outflow pathway region with respect to each of these tomographic images, and the intraocular pressure acquired at a substantially same date and time as the date and time when this tomographic image is acquired, into the external storage unit 400 in association with one another. Further, the present invention also includes an embodiment in which the display control unit 305 displays the measured value of the aqueous humor outflow pathway region that is measured with respect to each of these tomographic images captured at substantially same image-capturing positions, and the intraocular pressure acquired at the substantially same date and time as the date and time when this tomographic image is acquired, on the display unit 500 in association with each other as a graph like an example illustrated in FIG. 9C. For example, in FIG. 9C, measured values Md and intraocular pressures Mo regarding the aqueous humor outflow pathway region at the substantially same sites that are measured on days before, immediately after, and several months after a glaucoma surgery are displayed in association with each other, which facilitates confirmation of a treatment effect of the glaucoma surgery. In this display, a measured value Bd and an intraocular pressure Bo before the surgery are set as base lines (reference values).

Each of the above-described exemplary embodiments is an example in which the present invention is embodied as the image processing apparatus. However, embodiments of the present invention are not limited only to the image processing apparatus. For example, the present invention can be embodied as a system, an apparatus, a method, a program, a storage medium, or the like. More specifically, the present invention may be applied to a system constituted by a plurality of devices, or may be applied to an apparatus constituted by a single device.

Further, the present invention can also be realized by performing the following processing. Specifically, the present invention can also be realized by processing for supplying software (a program) capable of realizing the functions of the above-described exemplary embodiments to a system or an apparatus via a network or various kinds of recording media, and causing a computer, a central processing unit (CPU), a micro processing unit (MPU), or the like of this system or apparatus to read out and execute the program.

### Other Embodiments

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)(trademark)), a flash memory device, a memory card, and the like.

While the present invention has been described with reference to exemplary embodiments, it is to he understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-236742, filed Dec. 3, 2015, which is hereby incorporated by reference herein in its entirety.

1. An image processing apparatus comprising:
an acquisition unit configured to acquire a tomographic image containing an aqueous humor outflow pathway region that includes at least one of a Schlemm's canal and a collector channel in an anterior ocular segment of a subject's eye; and
a generation unit configured to generate an image with the aqueous humor outflow pathway region emphasized or extracted therein based on a luminance value of the tomographic image.

2. The image processing apparatus according to claim 1, wherein the generation unit generates the image with the aqueous humor outflow pathway region emphasized or extracted therein based on a change in the luminance value of the tomographic image in a depth direction.

3. The image processing apparatus according to claim 2, wherein the generation unit generates the image with the aqueous humor outflow pathway region emphasized or extracted therein by performing spatial differentiation processing on the luminance value of the tomographic image in the depth direction.

4. The image processing apparatus according to claim 1, further comprising a determination unit configured to determine a region including at least a sclera as a partial region of the tomographic image,
wherein the generation unit generates the image with the aqueous humor outflow pathway region emphasized or extracted therein based on the luminance value inside the determined region.

5. The image processing apparatus according to claim 4, wherein the generation unit performs flattening processing on the determined region to flatten a surface of the sclera, and generates the image with the aqueous humor outflow pathway region emphasized or extracted therein based on the luminance value inside the region on which the flattening processing has been performed.

6. The image processing apparatus according to claim 5, wherein the generation unit generates a plurality of two-dimensional images each acquired by projecting, on a plane intersecting with the depth direction, a value indicating a change in a luminance value of a different region in the depth direction inside the region on which the flattening processing has been performed.

7. The image processing apparatus according to claim 6, wherein the generation unit generates the image with the aqueous humor outflow pathway region emphasized or extracted therein by combining the generated plurality of two-dimensional images with a different display manner assigned to the aqueous humor outflow pathway region in each of the plurality of two-dimensional images.

8. The image processing apparatus according to claim 7, further comprising:
a display control unit configured to cause a two-dimensional image acquired by combining the plurality of two-dimensional images to be displayed on a display unit; and
an input unit configured to input a position specified by a user in the displayed two-dimensional image,
wherein the generation unit generates a curved planar image along the input position.

9. The image processing apparatus according to claim 5, wherein the generation unit generates the image with the aqueous humor outflow pathway region emphasized or extracted therein by generating a three-dimensional image based on a value acquired by calculating a second-order differential in the depth direction inside the region on which the flattening processing has been performed.

10. The image processing apparatus according to claim 9, further comprising a display control unit configured to cause a plurality of two-dimensional images at positions different in the depth direction that constitutes the generated three-dimensional image to be displayed continuously along the depth direction on a display unit.

11. The image processing apparatus according to claim 6, further comprising a display control unit configured to cause the generated plurality of two-dimensional images to be displayed continuously along the depth direction on a display unit.

12. The image processing apparatus according to claim 1, wherein the generation unit generates the image with the aqueous humor outflow pathway region emphasized or extracted therein by generating a two-dimensional image acquired by projecting a value indicating a change in the luminance value of the tomographic image in the depth direction.

13. The image processing apparatus according to claim 1, wherein the generation unit generates the image with the aqueous humor outflow pathway region emphasized or extracted therein by generating a three-dimensional image based on a value acquired by calculating a second-order differential processing on the luminance value of the tomographic image in the depth direction.

14. The image processing apparatus according to claim 1, further comprising a display control unit configured to cause the generated image to be displayed on a display unit,
wherein the generation unit generates a plurality of two-dimensional images each acquired by projecting, on a plane intersecting with the depth direction, a value indicating a change in a luminance value of a different region of the tomographic image in the depth direction, and
wherein the display control unit causes the generated plurality of two-dimensional images to be displayed continuously along the depth direction on the display unit.

15. The image processing apparatus according to claim 1, further comprising an identifying unit configured to identify a predetermined region from the aqueous humor outflow pathway region in the generated image,
wherein the identifying unit identifies a branch running on a deep scleral portion and a corneal central side as a Schlemm's canal region from the aqueous humor outflow pathway region.

16. The image processing apparatus according to claim 15, wherein the identifying unit identifies a branch connected to the identified Schlemm's canal region in the tomographic image as a collector channel region.

17. The image processing apparatus according to claim 1, further comprising a measurement unit configured to measure a shape of the aqueous humor outflow pathway region based on the generated image.

18. The image processing apparatus according to claim 17, further comprising a lesion detection unit configured to detect a lesion candidate with respect to the aqueous humor outflow pathway region based on a measured value regarding the shape of the aqueous humor outflow pathway region.

19. An image processing method comprising:
one or more processors; and
memory coupled to the one or more processors and storing instructions, the instructions, when executed by the one or more processors, cause the one or more processors to perform operations comprising:
acquiring a tomographic image containing an aqueous humor outflow pathway region that includes at least one of a Schlemm's canal and a collector channel in an anterior ocular segment of a subject's eye; and
generating an image with the aqueous humor outflow pathway region emphasized or extracted therein based on a luminance value of the tomographic image.

20. A non-transitory computer-readable storage medium storing a program for causing a computer to perform each operation in the image processing method according to claim 19.
